(19) United States
(12) Patent Application Publication
(10) Pub. No.: US 2017/0130277 A1
(43) Pub. Date: May 11, 2017

(54) BIOMARKER FOR COLORECTAL CANCER

(71) Applicants: STICHTING VU-VUMC, Amsterdam (NL); INTERNA TECHNOLOGIES B.V., Nijmegen (NL)

(72) Inventors: Gerrit Albert MEIJER, Amsterdam (NL); Begoña DIOSDADO, Amsterdam (NL); Lisette M. TIMMER, Amsterdam (NL); Edwin Pieter Johan Gerard CUPPEN, Bilthoven (NL); Eugene BEREZIKOV, Bedum (NL); Francesco CERISOLI, Rotterdam (NL); Roeland Quirinus Jozef SCHAAPVELD, Bussum (NL)

(73) Assignees: STICHTING VU-VUMC, Amsterdam (NL); INTERNA TECHNOLOGIES B.V., Nijmegen (NL)

(21) Appl. No.: 15/319,452

(22) PCT Filed: Jun. 19, 2015
(86) PCT No.: PCT/NL2015/050449

§ 371 Date: Dec. 16, 2016

(30) Foreign Application Priority Data
Jun. 19, 2014 (NL) 2013039

(51) Int. Cl. C12Q 1/68

(52) U.S. Cl. CPC C12Q 1/6886; C12Q 2600/178; C12Q 2600/158; C12Q 2600/16

(57) ABSTRACT

Methods of diagnosing colorectal cancer and precursors thereof using miRNA biomarkers are disclosed, together with kits and devices for detecting the biomarkers, and uses of the biomarkers. The biomarkers described are reliably detected in stool and demonstrate significantly different expression levels in colorectal cancer patients when compared to colorectal cancer negative patients.

### FIELD OF THE INVENTION

The present invention relates to methods of diagnosing and treating colorectal cancer or a precursor of colorectal cancer, use of a biomarker in such methods, and corresponding devices, arrays and kits.

### BACKGROUND OF THE INVENTION

Colorectal cancer (CRC) is a major health burden. It arises from epithelium that forms the inner lining of the bowel wall. In a multistep process, via a premalignant stage called adenoma, neoplasia can progress towards cancer. Underlying this neoplastic process are disruptions of critical biological processes, the status of which is reflected by changes at the DNA, RNA and protein level (Nature 2012; 487:330-337; N Engl J Med 2009; 361:2449-2460). Screening, comprising early detection and removal of lesions, is the best approach to decrease the high number of CRC related deaths (Eur J Cancer 2013; 49:1374-1403; Ann Oncol 2013; 24(8) 1963-1972; N Engl J Med 2013; 369:1106-1114). Multiple options exist, but most countries applying programmatic CRC screening have selected a stool test over colonoscopy (Neth J Med 2011; 69:112-119). Nevertheless, the performance of hemoglobin based stool tests leaves room for improvement, which may come from molecular markers (Gastroenterology 2012; 142:248-256; Nat Rev Cancer 2005; 5:199-209; Gastroenterology 2013; 144:918-925; Int J Colorectal Dis 2012; 27:1657-1664). For this purpose, mostly tumor derived DNA and tumor associated proteins present in stool have been explored (Clin Colorectal Cancer 2011; 10:8-23). Yet microRNAs (miRNAs) are an attractive alternative. First and foremost these biomarkers should have a high sensitivity for cancer, but also for relevant precursor lesions (adenomas). It is known that the vast majority of adenomas do not progress to cancer (Sillars-Hardebol, A. H., Carvalho, B., van Engeland, M., Fijneman, R. J. A., & Meijer, G. A. (2011). The adenoma hunt in colorectal cancer screening: defining the target. The Journal of Pathology, 226(1), 1-6. doi:10.1002/path.3012), and thus are not harmful for the patient. Therefore, biomarkers should be specific for the about five percent of high-risk adenomas that do progress to cancer. In clinical practice, the class of advanced adenomas is considered to reflect these high-risk lesions, as opposed to non-advanced adenomas. Which miRNAs discriminate colorectal cancer and advanced adenomas from non-advanced adenomas remains to be resolved.

Currently, over 2500 mature human miRNAs have been annotated (miRBase release 20, June 2013) (Nucleic Acids Res 2004; 32:D109-D111; Nucleic Acids Res 2006; 34:D140-D144; Nucleic Acids Res 2008; 36:D154-D158; Nucleic Acids Res 2011; 39:D152-D157). MiRNA expression levels are highly tissue specific, but within a given tissue type, they also differ between different disease states, e.g. normal and cancer (Expert Rev Mol Diagn 2010; 10:435-444). Differential expression pattern of miRNAs in plasma from CRC patients and healthy controls has been reported (Gut 2009; 58:1375-1381), and levels of elevated miRNAs significantly decreased in post-operative plasma samples when compared to pre-operative samples in CRC patients. This provides a strong indication of a direct relation between elevated miRNA levels and the presence of disease.

A specific stool-based miRNA expression signature for detecting CRC would hold potential for a CRC screening test. Due to the existence of a direct interface between the tumor and stool, miRNAs present in stool may therefore be suitable biomarkers for use in non-invasive screening tests.

The present invention seeks to overcome some or all of the above-mentioned problems.

### SUMMARY OF THE INVENTION

According to a first aspect of the present invention there is provided a method of detecting colorectal cancer or a precursor of colorectal cancer in a subject, the method comprising:
obtaining a stool sample from the subject;
analyzing the stool sample for the increased expression of hsa-miR-223 relative to a reference sample, wherein the increased expression of hsa-miR-223 relative to the reference sample is indicative that the individual is suffering from colorectal cancer or a precursor of colorectal cancer.

According to a second aspect of the present invention there is provided a method of predicting the likelihood that a subject is at risk of suffering from or is suffering from advanced adenoma and/or colorectal cancer, the method comprising:
obtaining a stool sample from the subject;
analyzing the stool sample for the increased expression of hsa-miR-223 relative to a reference sample, wherein the increased expression of hsa-miR-223 relative to the reference sample is indicative that the individual is at risk of suffering from or is suffering from advanced adenoma and/or colorectal cancer.

According to a third aspect of the present invention there is provided a device configured for detecting advanced adenoma and/or colorectal cancer in a subject, the device comprising means for measuring the level of hsa-miR-223 in a stool sample obtained from the subject, and means for displaying the results of the measurement, the device being present in association with instructions for use in any of the diagnostic or predictive methods described herein.

According to a fourth aspect of the present invention there is provided a kit for measuring the level of hsa-miR-223 in a stool sample obtained from a subject, the kit comprising: (a) an hsa-miR-223 binding agent that selectively binds hsa-miR-223, (b) packaging materials and instructions for measuring hsa-miR-223 in a sample, the kit being present in association with instructions for use in any of the diagnostic or predictive methods described herein.

According to a fifth aspect of the present invention there is provided an array for detecting colorectal cancer, the array comprising a binding agent specific to hsa-miR-223.

According to a sixth aspect of the present invention, there is provided a use of hsa-miR-223 as a biomarker in detecting colorectal cancer.

The present inventors have identified that the miRNA hsa-miR-223 can be detected in stool samples at significantly different levels between CRC sufferers or advanced adenoma sufferers and healthy individuals. Identification of a new biomarker which can be reliably detected in stool and can distinguish between CRC sufferers or advanced adenoma sufferers and healthy individuals enables the development of new diagnostic tests, assays and arrays, as well as methods of treatment based on the results of the diagnoses.

Further, non-essential embodiments of the invention are the subject of the dependent claims.

### BRIEF DESCRIPTION OF THE FIGURES

The present invention will now be described by way of example only, and without limitation, by reference to the following Figures, in which:

FIG. 1 shows a flow chart of the complete study design, starting with the genome-wide discovery phase identifying miRNAs involved in colorectal adenoma to carcinoma progression by Next-Generation Sequencing (NGS—on pooled samples). The following three phases were executed by RT-qPCR, comprising of validation of the differentially expressed miRNAs on individual tissue level, testing the remaining miRNAs on stool specimens and validation of these miRNAs in stool specimens;

FIGS. 2A and 2B show boxplots representing the normalized expression levels of 21 miRNAs in a validation series of colorectal adenoma and carcinoma tissue samples. These 21 miRNAs were selected out of the originally 57 miRNAs discovered by Next Generation Sequencing (NGS) in a pooled series of colorectal adenomas and carcinomas;

FIG. 3 shows boxplots representing the normalized levels of expression for eight miRNAs in stool samples from healthy controls and patients with non-advanced adenomas, advanced adenomas and colorectal cancer.

FIG. 4 shows ROC analyses of miR-223 in stool as screening test against colonoscopy negative controls (n=76). Continuous lines represent the whole study population (WSP), i.e. all ages, and dotted lines represent the screening target population age groups (Scr.P), i.e. 55-75 years. (A) All cancers (n=110), WSP: AUC 0.86, specificity (spec.) 95%, sensitivity (sens.) 51%; ScrP: AUC 0.90, spec. 95%, sens. 74%. (B) Stage I & II cancers (n=36), WSP: AUC 0.82, spec. 95%, sens. 50%; ScrP: AUC 0.84, spec. 95%, sens. 64%. (C) Advanced adenomas (n=39), WSP: AUC 0.52, spec. 95%, sens. 5%; ScrP: AUC 0.63, spec. 95%, sens. 23%. (D) Screen-relevant lesions (n=75), i.e. advanced adenomas and stage I & II colorectal cancer, WSP: AUC 0.67, spec. 95%, sens. 23%; ScrP: AUC 0.74, spec. 95%, sens. 43%;

FIG. 5 shows ROC analyses of miR-223 in stool combined with FIT in colorectal cancer versus colonoscopy negative controls. At 95% specificity, FIT alone has 82% sensitivity in the whole study population (all ages, AUC 0.90) and 70% sensitivity in the screening target population (age 55-75, AUC 0.85). Combining miR-223 with FIT showed at 95% specificity an 88% sensitivity in the whole study population (AUC 0.94) and 73% sensitivity in the screening population (AUC 0.91); and

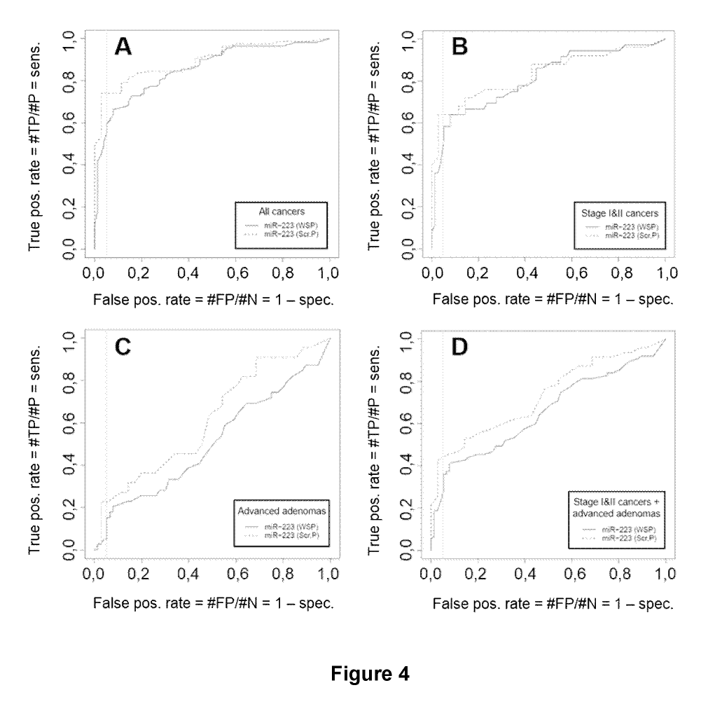

FIG. 6 shows ROC analyses of up-regulated miRNAs miR-223 and miR-214 in colorectal cancer patients versus colonoscopy negative controls. (A) Combining miR-223 and miR-214 showed at 95% specificity, 54.5% sensitivity in the whole study population (AUC 0.87) and 74% sensitivity in the screening population (AUC 0.90). (B) MiR-214 detection alone showed at 95% specificity, 10.0% sensitivity (AUC 0.62) in the whole study population and 5.2% sensitivity (AUC 0.61) in the screening population.

### DETAILED DESCRIPTION OF THE INVENTION

### Colorectal Cancer

The most common colorectal cancer cell type is adenocarcinoma which accounts for 95% of cases. Other, rarer types include lymphoma and squamous cell carcinoma. Colorectal adenocarcinoma arises from precursor lesions called adenomas, of which only a minority progress to cancer. Adenomas that progress to cancer are referred to as high risk adenomas.

The approach of measuring molecules directly in stool is of significance to reveal biomarkers that are stable in the fecal environment and detectable in the background of bacterial- and food-related molecules.

The present invention is advantageously used for screening for colorectal cancer, for example, adenocarcinoma found in the colon or rectum. However, the methods of the invention should not be considered as being limited solely to the detection of colonic adenocarcinomas. Rather, the methods of the invention are also useful in the detection of advanced or high-risk colonic adenomas, thus enabling the identification of an individual at risk of developing colorectal cancer due to the presence of an advanced or a high-risk adenoma.

References herein to screening for colorectal cancer thus may include screening for advanced colorectal adenomas and high-risk adenomas as well as colorectal adenocarcinoma.

It is also expected that the biomarker identified by the present invention may also find application for the diagnosis of adenocarcinomas present higher up the gastrointestinal tract.

Thus, the present invention may also provide a method for screening for gastrointestinal disease or gastrointestinal cancer, the method comprising: screening a stool sample obtained from an individual for the increased expression of hsa-miR-223 relative to a reference sample, wherein the increased expression of hsa-miR-223 relative to the reference sample is indicative that the patient is at risk of suffering from or is suffering from gastrointestinal disease or gastrointestinal cancer.

### Sample

The sample for analysis is a stool sample obtained from a subject or individual. The subject or individual is preferably a human subject. In one embodiment, the subject has or is suspected of having adenoma. In one embodiment, the subject has or is suspected of having non-advanced adenoma. In one embodiment, the subject has or is suspected of having advanced adenoma. In one embodiment, the subject has or is suspected of having colorectal cancer.

The sample may be prepared by any conventional method for extracting total RNA from a biological sample, particularly a stool sample. For example, the stool sample may be mixed with stool stabilization buffer (Exact Sciences, Madison, Wis., USA) immediately after defecation, and processed to a final stool:buffer w/v ratio of 1:7 within 72 hours, and stored at −80° C. until use. One exemplary means for extracting total RNA from a stool sample is by using TRIzol (Invitrogen, Carlsbad, Calif., USA). The total RNA so obtained may be cleaned up via an ethanol precipitation.

Determining the Expression Levels of miRNAs

Despite the complexity of stool material, the present invention has successfully detected several miRNAs in stool that are predictive of the presence of colorectal cancer and/or adenoma. In particular, the identification of hsa-miR-223 as a reliable biomarker for the detection of colorectal cancer, in addition to distinguishing colorectal cancer from non-advanced adenoma and advanced adenoma enables the diagnostic and predictive methods described herein.

Thus, as described in detail herein, the present inventors have identified that the increased expression of hsa-miR-223 relative to a reference sample is indicative that the individual from which the sample was obtained is suffering from colorectal cancer or a precursor of colorectal cancer, for example advanced adenoma.

Furthermore, the present inventors have identified that the increased expression of hsa-miR-214 relative to a reference sample may also be indicative that the individual from which the sample was obtained is suffering from colorectal cancer or a precursor of colorectal cancer, for example advanced adenoma. Furthermore, the present inventors have identified that the decreased expression of hsa-miR-200b or hsa-miR-141 relative to a reference sample may also be indicative that the individual from which the sample was obtained is suffering from colorectal cancer or a precursor of colorectal cancer, for example advanced adenoma.

Thus, any of the methods, devices and kits described herein with reference to hsa-miR-223 may be adapted to detect or measure the expression levels of one or more of hsa-miR-200b, hsa-miR-141, and hsa-miR-214 relative to a reference sample.

The miRNA hsa-miR-223 is a miRNA of 110 nucleotides with the following primary sequence: ccuggccucc ugcagugcca cgcuccgugu auuugacaag cugaguugga cacuccaug gguagagugu caguuuguca aauaccccaa gugcggcaca ugcuuaccag (SEQ ID NO:1) (Accession number MI0000300 in miRBase release 20, June 2013).

Sequences of all miRNAs disclosed in the present application, including the full length sequences (for example stem-loop sequences) and mature 3p and mature 5p sequences can be found in miRBase release 20, June 2013.

The following methods and assays form part of the present disclosure and may constitute aspects or embodiments of the present invention.

A method of diagnosing colorectal cancer in an individual, comprising measuring the amount of one or more of hsa-miR-223, hsa-miR-200b, hsa-miR-141, and hsa-miR-214 present in a stool sample obtained from the individual, wherein the increased expression of hsa-miR-223 and/or hsa-miR-214 or the decreased expression of hsa-miR-200b and/or hsa-miR-141, relative to a reference sample is indicative that the individual is suffering from colorectal cancer.

A method of detecting colorectal cancer or a precursor of colorectal cancer in a subject, the method comprising obtaining a stool sample from the subject; and analyzing the stool sample for the increased expression of one or both of hsa-miR-223 and hsa-miR-214 or the decreased expression of one or both of hsa-miR-200b and/or hsa-miR-141 relative to a reference sample.

A method of predicting the likelihood that a subject is at risk of suffering from or is suffering from advanced adenoma and/or colorectal cancer, the method comprising: obtaining a stool sample from the subject; and analyzing the stool sample for the increased expression of one or both of hsa-miR-223 and hsa-miR-214 or the decreased expression of one or both of hsa-miR-200b and/or hsa-miR-141 relative to a reference sample.

An assay comprising: contacting a stool sample obtained from a subject with a detectable binding agent specific for hsa-miR-223; washing the sample to remove unbound binding agent; measuring the intensity of the signal from the bound, detectable binding agent; comparing the measured intensity of the signal with a reference value and identifying the subject as having an increased probability of having colorectal cancer or a precursor to colorectal cancer if the measured intensity is increased relative to the reference value.

An assay comprising: extracting total RNA from a stool sample obtained from an individual, subjecting the extracted total RNA to size exclusion fractionation to isolate small RNA molecules having less than 150 nucleobases; and sequencing the isolated small RNA molecules to determine the amount of one or both of hsa-miR-223 or hsa-miR-214 in the stool sample relative to a reference sample; and identifying the individual as having an increased probability of having colorectal cancer or a precursor to colorectal cancer if the measured intensity is increased relative to the reference value.

An assay comprising: extracting total RNA from a stool sample obtained from an individual, subjecting the extracted total RNA to size exclusion fractionation to isolate small RNA molecules having less than 150 nucleobases; and sequencing the isolated small RNA molecules to determine the amount of one or both of hsa-miR-200b or hsa-miR-141 in the stool sample relative to a reference sample; and identifying the individual as having an increased probability of having colorectal cancer or a precursor to colorectal cancer if the measured intensity is decreased relative to the reference value.

An assay comprising: extracting total RNA from a stool sample obtained from an individual, amplifying the extracted total RNA by RT-qPCR using an LNA primer set including LNA primers specific to hsa-miR-223 or hsa-miR-214; determining the amount of hsa-miR-223 and/or hsa-miR-214 in the stool sample relative to a reference sample; and identifying the individual as having an increased probability of having colorectal cancer or a precursor to colorectal cancer if the measured intensity is increased relative to the reference value.

An assay comprising: extracting total RNA from a stool sample obtained from an individual, amplifying the extracted total RNA by RT-qPCR using an LNA primer set including LNA primers specific to hsa-miR-200b or hsa-miR-141; determining the amount of hsa-miR-200b and/or hsa-miR-141 in the stool sample relative to a reference sample; and identifying the individual as having an increased probability of having colorectal cancer or a precursor to colorectal cancer if the measured intensity is decreased relative to the reference value.

In one embodiment of the methods and assays described herein, any miRNA present in the test sample is labelled with a detectable moiety. In one embodiment, any miRNA present in the control sample is labelled with a detectable moiety (which may be the same or different from the detectable moiety used to label the test sample).

A “detectable moiety” is one which may be detected and the relative amount and/or location of the moiety (for example, the location on an array) determined.

Detectable moieties are well known in the art. A detectable moiety may be a fluorescent and/or luminescent and/or chemiluminescent moiety which, when exposed to specific conditions, may be detected. For example, a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be a radioactive label, which may be incorporated by methods well known in the art.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

In one embodiment, the methods described herein may utilise a sequencing methodology in order to detect the increased expression of hsa-miR-223 and/or hsa-miR-214; or the decreased expression of hsa-miR-200b and/or hsa-miR-141. In one embodiment, the step of detecting the expression level of an miRNA such as hsa-miR-223 in the methods described herein may comprise subjecting the total RNA from a sample obtained from a subject to a sequencing step. The sequencing step may comprise a “next-generation” sequencing method, for example pyrosequencing, sequencing by synthesis (from Illumina), sequencing by ligation (SOLiD sequencing from Applied Biosystems) or single molecule real-time sequencing (from Pacific Biosciences). In one embodiment, the total RNA may be subjected to reverse transcription to form the corresponding cDNA prior to sequencing, which may then be optionally amplified by PCR.

In one embodiment, the methods described herein may utilise an amplification methodology in order to detect the increased expression of hsa-miR-223 or hsa-miR-214. In one embodiment, the step of detecting the increased expression of hsa-miR-223 or hsa-miR-214 in the methods described herein may comprise subjecting the total RNA from a sample obtained from a subject to an amplification step. In one embodiment, the step of detecting the decreased expression of hsa-miR-200b or hsa-miR-141 in the methods described herein may comprise subjecting the total RNA from a sample obtained from a subject to an amplification step. In one embodiment, the total RNA may be subjected to reverse transcription to form the corresponding cDNA prior to amplification. In one embodiment, the total RNA is amplified without transcription to the corresponding cDNA. In one embodiment, the total RNA is subjected to amplification by PCR (polymerase chain reaction). The PCR reaction may be any type of PCR reaction, for example a RT-qPCR, an emulsion PCR, or the related QuARTS technology (Quantitative Allele-Specific Real-time Target and Signal amplification). The PCR may be a real-time PCR. In one embodiment, the real-time PCR is a quantitative real-time PCR, i.e. RT-qPCR. In one embodiment, the amplification reaction uses primers specific to hsa-miR-223 or any other miRNA listed herein. In other words, the amplification reaction uses binding agents which are selective for hsa-miR-223 or any other miRNA listed herein. In one embodiment, the amplification reaction uses LNA-based primers or binding agents which are selective for hsa-miR-223 or any other miRNA listed herein. Unless otherwise stated, references to “selective for” are to be understood as meaning that there is sufficient complementarity along the sequence length of the primer that the primer will hybridise to the target (hsa-miR-223). It will be understood that hybridisation can still occur with a number of mismatches in primary sequence through bulges and loops.

The amount and/or concentration of hsa-miR-223 or any other miRNA listed herein in the sample are compared with the amount and/or concentration of hsa-miR-223 or any other miRNA listed herein as determined in a control or reference sample. Such comparison will be based on the information obtained in the above determination of the amount and/or concentration of hsa-miR-223. Analogous comparisons may be made for any other miRNA listed herein. The data or information can be present in either written or electronic form, i.e. on a suitable storage medium. The comparison can either be performed manually and individually, e.g. visually by the attending physician or the scientist in the diagnostic facility, or done by a suitable machine, for example a computer equipped with suitable software. Such equipment is preferred for routine screening. High-throughput environments (e.g. assemblies) for such methods are known to the person skilled in the art and also described in the standard literature.

### Reference Levels

The reference level of hsa-miR-223 or any other miRNA listed herein for establishing the expectation (or lack of expectation) that the subject has or is at risk of having colorectal cancer or a precursor thereof may be established from population studies of different categories of colorectal cancer or advanced or non-advanced adenoma and different ages of patients at disease onset, so that the sensitivity and specificity of the assay can be set as high as possible for the patient's personal parameters.

The reference level may be set having regard to the patient's own medical history and/or other relevant factors, for example age. These factors may be taken into account together with the population studies data to establish, for each individual patient or for groups of patients, the reference level for use to predict the likelihood of the subject having colorectal cancer or a precursor to colorectal cancer.

The term “reference” thus refers to a reference value, or range of values, which may be obtained from a suitable number of subjects selected from colonoscopy-negative controls, colorectal cancer-positive patients, advanced adenoma-positive patients and/or non-advanced adenoma-positive patients.

The reference value(s) can be stored on, for example, a computer or PDA device to permit comparison with a value obtained from a subject using the methods described herein.

### Binding Agents

In one embodiment, the methods and kits described herein use a binding agent that selectively binds to hsa-miR-223 to determine the presence or increased expression of hsa-miR-223. In other embodiments, the methods and kits described herein may also use binding agents that selectively bind one or more of hsa-miR-200b, hsa-miR-141 or hsa-miR-214. Binding agents (also referred to as binding molecules) can be selected from a library, based on their ability to bind a given motif, as discussed below.

In one embodiment the binding agent is an antibody. The fecal immunochemical test (FIT) comprises an antibody based screening assay, and so an antibody based screening assay for hsa-miR-223 or any other miRNA described herein may provide a complementary screen which can be readily incorporated into the existing FIT assay.

Thus, in one embodiment, the binding agent may be an antibody or a fragment thereof. A fragment may contain one or more of the variable heavy (V_{H}) or variable light (V_{L}) domains. For example, the term antibody fragment includes Fab-like molecules (Better et al Science 1988; 240, 1041); Fv molecules (Skerra et al Science 1988; 240, 1038); single-chain Fv (ScFv) molecules where the V_{H} and V_{L} partner domains are linked via a flexible oligopeptide (Bird et al Science 1988; 242,423; Huston et al Proc. Natl. Acad. Sci. USA 1988; 85, 5879) and single domain antibodies (dAbs) comprising isolated V domains (Ward et al Nature 1989; 341,544).

The term “antibody variant” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecule capable of binding to an antigen in an immunoassay format that is known to those skilled in the art.

A general review of the techniques involved in the synthesis of antibody fragments which retain their specific binding sites is to be found in Winter & Milstein Nature 1991; 349, 293-299.

In one embodiment, the antibody or fragment thereof is a recombinant antibody or fragment thereof (such as an scFv). By “ScFv molecules” it is meant molecules wherein the V_{H} and V_{L} partner domains are linked via a flexible oligopeptide.

The advantages of using antibody fragments, rather than whole antibodies, are several-fold. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” it is meant that the said antibodies and F(ab′)₂ fragments have two antigen combining sites. In contrast, Fab, Fv, ScFv and dAb fragments are monovalent, having only one antigen combining site.

The antibodies may be monoclonal or polyclonal. Suitable antibodies may be prepared by known techniques and need no further discussion.

Additionally or alternatively the binding agent may be an aptamer. Suitable aptamers may be prepared by known techniques and need no further discussion.

In one embodiment, the methods and arrays may utilise a binding agent in the form of a nucleic acid complementary to hsa-miR-223 or any of hsa-miR-200b, hsa-miR-141, and hsa-miR-214. Unless otherwise stated, the term “complementary” insofar as it relates to nucleic acid hybridisation is intended to mean that there is sufficient sequence complementarity to the primary sequence of the target, e.g. hsa-miR-223, that hybridisation occurs. That is to say, the nucleic acid complementary to hsa-miR-223 need not have 100% complementarity to hsa-miR-223 along the sequence length. For example, the binding agent may have 95% complementarity along the primary sequence length, or 90% complementarity along the sequence length. In one embodiment, the nucleic acid based binding agent is selected from DNA, RNA or synthetic nucleic acid analogues such as PNA or LNA. The nucleic acid based binding agent may be immobilised on the array and hybridisation to the target, for example hsa-miR-223, may be detected by any method known in the art. For example, the nucleic acid based binding agent may detect the presence of or increased expression of hsa-miR-223 based on optical techniques (e.g. fluorescence), electrochemical techniques, electronic techniques, piezoelectric techniques, gravimetric techniques or pyroelectric techniques.

Thus, the binding agents useful in the present invention may comprise one or more primer sets which selectively bind to hsa-miR-223 for the purpose of one or more of reverse transcription, amplification and/or sequencing. In one embodiment, the binding agents useful in the present invention may comprise binding agents which selectively bind to hsa-miR-223, wherein the binding agents comprise LNA PCR primers. In one embodiment, the binding agents useful in the present invention may comprise binding agents in the form of the following LNA PCR primers for amplifying hsa-miR-223: 5′-TGGGGTATTTGACAAACTGACA-3′ (SEQ ID NO:2) and 5′-AACTCAGCTTGTCAAATACACG-3′ (SEQ NOD NO:3).

### Arrays

In one embodiment, the methods of the present invention may use binding agents as described previously and be carried out on an array.

Arrays per se are well known in the art. Typically they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution.

The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising antibodies, complementary nucleic acid strands and other suitable molecules and/or conducting a binding assay.

The immobilisation processes are well known in the art and generally consist of cross-linking, covalently binding or physically adsorbing an antibody or complementary nucleic acid strand to the solid support. By using well-known techniques, such as contact or non-contact printing, masking or photolithography, the location of each spot can be defined.

Once suitable binding molecules (discussed above) have been identified and isolated, the skilled person can manufacture an array using methods well known in the art of molecular biology.

### Compounds and Methods for Treating CRC

The identification of hsa-miR-223, hsa-miR-200b, hsa-miR-141 and hsa-miR-214 as markers allows not only the detection of advanced colonic adenomas and colonic adenocarcinomas (colorectal cancer), but enables also methods of treating colorectal cancers as described herein, and also provides for compounds for use in methods of treating colorectal cancers also as described herein.

For example, the present invention may enable a method of treating or preventing colorectal cancer in a subject, the method comprising: obtaining a stool sample from the subject; analyzing the stool sample to identify the increased expression of hsa-miR-223 or hsa-miR-214 relative to a reference sample; and administering a cancer treatment selected from surgical resection, radiation therapy and chemotherapy to the subject when the increased expression of hsa-miR-223 or hsa-miR-214 relative to a reference sample is identified.

Alternatively, the present invention may enable a method of treating advanced adenoma in a subject, the method comprising: obtaining a stool sample from the subject; analyzing the stool sample to identify the increased expression of hsa-miR-223 or hsa-miR-214 relative to a reference sample; and administering a treatment selected from surgical resection, radiation therapy and chemotherapy to the subject when the increased expression of hsa-miR-223 or hsa-miR-214 relative to a reference sample is identified.

Alternatively, the present invention provides a chemotherapeutic agent for use in a method of treating colorectal cancer in a subject, the method comprising: obtaining a stool sample from the subject; analyzing the stool sample to identify the increased expression of hsa-miR-223 or hsa-miR-214 relative to a reference sample; and administering the chemotherapeutic agent to the subject when the increased expression of hsa-miR-223 or hsa-miR-214 relative to a reference sample is identified.

Furthermore, the present invention may enable a method of treating or preventing colorectal cancer in a subject, the method comprising: obtaining a stool sample from the subject; analyzing the stool sample to identify the decreased expression of hsa-miR-200b or hsa-miR-141 relative to a reference sample; and administering a cancer treatment selected from surgical resection, radiation therapy and chemotherapy to the subject when the decreased expression of hsa-miR-200b or hsa-miR-141 relative to a reference sample is identified.

Alternatively, the present invention may enable a method of treating advanced adenoma in a subject, the method comprising: obtaining a stool sample from the subject; analyzing the stool sample to identify the decreased expression of hsa-miR-200b or hsa-miR-141 relative to a reference sample; and administering a treatment selected from surgical resection, radiation therapy and chemotherapy to the subject when the decreased expression of hsa-miR-200b or hsa-miR-141 relative to a reference sample is identified.

Alternatively, the present invention provides a chemotherapeutic agent for use in a method of treating colorectal cancer in a subject, the method comprising: obtaining a stool sample from the subject; analyzing the stool sample to identify the decreased expression of hsa-miR-200b or hsa-miR-141 relative to a reference sample; and administering the chemotherapeutic agent to the subject when the decreased expression of hsa-miR-200b or hsa-miR-141 relative to a reference sample is identified.

While early diagnosis of advanced adenoma or colorectal cancer often allows for curative surgical removal of the polyps or tumour, later diagnosis may result in a (chemo)therapeutic treatment instead. Therapeutic agents used to treat colorectal cancer include monoclonal antibodies, small molecule inhibitors and chemotherapeutic agents.

Typical therapeutic monoclonal antibodies include but are not limited to bevacizumab, cetuximab or panitumumab. Typical small molecule inhibitors include but are not limited to erlotinib, sorafenib or alisertib. Typical chemotherapeutic agents include but are not limited to 5-FU, capecitabine, irinotecan oxaliplatin, or leucovorin or any combination thereof. Combination therapies of, for example, a therapeutic monoclonal antibody and a small molecule inhibitor may be used. Thus, any combination of two or more of a monoclonal antibody, a small molecule inhibitor and a chemotherapeutic agent is envisaged.

### Kits for Performing the Method

In one aspect of the present invention, a kit for measuring the level of hsa-miR-223 or any of the miRNAs listed herein is provided. Specifically, the kit comprises (a) an hsa-miR-223 binding agent that selectively binds hsa-miR-223, and/or one or more binding agents that selectively bind one or more of hsa-miR-200b, hsa-miR-141 and hsa-miR-214; (b) packaging materials and instructions for measuring hsa-miR-223 and/or one or more of hsa-miR-200b, hsa-miR-141 and hsa-miR-214 in a sample, the kit being present in association with instructions for use according to any of the methods described herein.

The binding agent, for example an hsa-miR-223 binding agent may be any binding agent as hereinbefore described. For example, the hsa-miR-223 binding agent may comprise an antibody, an aptamer or a complementary nucleic acid sequence. In one example, the hsa-miR-223 binding agent comprises forward and reverse primers for an amplification reaction, for example a PCR reaction. The PCR reaction may be any type of PCR reaction, for example a RT-qPCR, an emulsion PCR, or the related QuARTS technology (Quantitative Allele-Specific Real-time Target and Signal amplification).

Thus, the kits of the present invention for use in any of the methods described herein may comprise one or more primer sets which selectively bind to hsa-miR-223 or any of the miRNAs listed herein for the purpose of one or more of reverse transcription, amplification and/or sequencing. In one embodiment, the kits of the present invention may comprise binding agents which selectively bind to one or more of hsa-miR-223, hsa-miR-200b, hsa-miR-141 and hsa-miR-214, wherein the binding agents comprise PCR primers. In one embodiment, the kits of the present invention may comprise binding agents which selectively bind to one or more of hsa-miR-200b, hsa-miR-141 and hsa-miR-214, wherein the binding agents comprise LNA-based PCR primers.

In one embodiment, the kits of the present invention may comprise binding agents in the form of the following LNA PCR primers for amplifying hsa-miR-223: 5′-TGGGGTATTTGACAAACTGACA-3′ (SEQ ID NO:2) and 5′-AACTCAGCTTGTCAAATACACG-3′ (SEQ ID NO:3). In one embodiment, the kits of the present invention may comprise binding agents in the form of the following LNA PCR primers for amplifying hsa-miR-200b: 5′-TCATCATTACCAGGCAGTATTA-3′ (SEQ ID NO:4) and 5′-TCCAATGCTGCCCAGTAAGATG-3′ (SEQ ID NO:5). In one embodiment, the kits of the present invention may comprise binding agents in the form of the following LNA PCR primers for amplifying hsa-miR-141: 5′-CCATCTTTACCAGACAGTGTTA-3′ (SEQ ID NO:6) and 5′-TCCAACACTGTACTGGAAGATG-3′ (SEQ ID NO:7). In one embodiment, the kits of the present invention may comprise binding agents in the form of the following LNA PCR primers for amplifying hsa-miR-214: 5′-ACTGCCTGTCTGTGCCTGCTGT-3′ (SEQ ID NO:8) and 5′-GCACAGCAAGTGTAGACAGGCA-3′ (SEQ ID NO:9).

### “Device”

The device configured for detecting advanced adenoma and/or colorectal cancer in a subject may include computer readable storage media holding data on the level of hsa-miR-223 or any of the miRNAs listed herein which correlates to an increase or decrease in expression predictive of non-advanced adenoma, advanced adenoma and/or colorectal cancer, and a data processing system that in use performs the comparison of the measured level of hsa-miR-223 or any of the miRNAs listed herein with the level of hsa-miR-223 or any of the miRNAs listed herein which correlates to an increase or decrease in expression predictive of non-advanced adenoma, advanced adenoma and/or colorectal cancer. The device may be configured for detecting non-advanced adenoma, advanced adenoma and/or colorectal cancer may be loaded with one or more binding agents selective for hsa-miR-223 or any of the miRNAs listed herein. In one embodiment, the device may be loaded with amplification primers selective for hsa-miR-223. The device may suitably be associated with an electronic display device and/or a printer, for displaying and/or printing the results of the comparison and derivation procedure.

The computer readable storage media suitably have computer readable instructions recorded thereon to define software modules including a determination system and a comparison module for implementing the method of the present invention on a computer. The computer implementation of the method may suitably comprise: (a) storing data derived from a stool sample obtained from a subject and which represents the level of hsa-miR-223 or any of the miRNAs listed herein in the sample, (b) comparing with the comparison module the data stored on the storage device with reference and/or control data, and to provide a retrieved content, and (c) displaying the retrieved content for the user, wherein the retrieved content is indicative that the subject has colorectal cancer or a precursor thereof, for example advanced adenoma, if the level of hsa-miR-223 or hsa-miR-214 in the sample is higher than the reference data, and wherein the retrieved content is indicative that the subject does not have colorectal cancer or a precursor thereof, for example advanced adenoma, if the level of hsa-miR-223 or hsa-miR-214 in the sample is the same level as or lower than the reference data; or wherein the retrieved content is indicative that the subject has colorectal cancer or a precursor thereof, for example advanced adenoma, if the level of hsa-miR-200b or hsa-miR-141 in the sample is lower than the reference data, and wherein the retrieved content is indicative that the subject does not have colorectal cancer or a precursor thereof, for example advanced adenoma, if the level of hsa-miR-200b or hsa-miR-141 in the sample is the same level as or higher than the reference data.

The data processing system that performs the comparison of the measured level of the miRNA, for example hsa-miR-223 with the level of hsa-miR-223 which correlates to colorectal cancer or a precursor thereof in the patient under investigation may suitably have computer systems for obtaining data on the level of hsa-miR-223 in the sample. The computer system may suitably comprise: (a) a determination system configured to receive data which represents the level of hsa-miR-223 in the sample obtained from a subject; (b) a storage device configured to store data output from the determination system; (c) a comparison module adapted to compare the data stored on the storage device with reference and/or control data, and to provide a retrieved content, and (d) a display module for displaying the retrieved content for the user, wherein the retrieved content is indicative that the has colorectal cancer or a precursor thereof, for example advanced adenoma, if the level of hsa-miR-223 in the sample is higher than the reference data, and wherein the retrieved content is indicative that the subject does not have colorectal cancer or a precursor thereof, for example advanced adenoma, if the level of hsa-miR-223 in the sample is the same level as or lower than the reference data.

The computer readable storage media and the computer system may constitute aspects of the present invention.

### “Comprising”

The term “comprising” and related terms herein is to be interpreted as embracing “consisting essentially of” and “consisting of”, these two expressions being interchangeable with “comprising” in all definitions and discussion in this patent in order to specify alternative extents of exclusion of unspecified elements additional to the recited elements.

The term “comprising” and related expressions means “including” and therefore leaves open the option of including unspecified elements, whether essential or not. The term “consisting essentially of” and related expressions permits the presence of elements that do not materially affect the basic and novel or functional characteristic(s) of that embodiment of the invention. The term “consisting of” and related expressions means “consisting only of” and therefore excludes any element not recited in that description of the embodiment.

### EXAMPLE

### Materials and Methods

### Study Design and Sample Collection

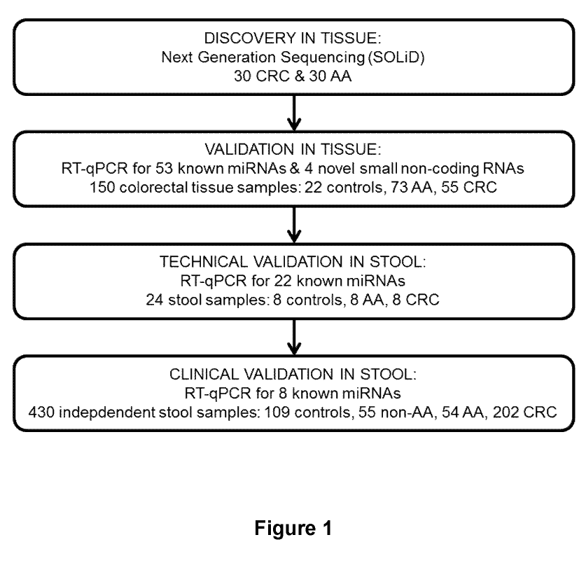

The current study was divided into three separate parts, involving three different, independent patient populations (FIG. 1). First, for a genome-wide discovery of miRNAs involved in colorectal adenoma to carcinoma progression, Next-Generation Sequencing (NGS) was carried out (SOLiD platform, Applied Biosystems) in colorectal tumor tissue specimens, which were pooled by category. Second, miRNAs differentially expressed between colorectal adenomas and carcinomas in the NGS data were validated in individual tumor tissue specimens by RT-qPCR (Exiqon, Vedbaek, Denmark). Third, miRNAs with validated differential expression in the second series were analyzed in stool samples of patients with colorectal cancers and adenomas, and compared to colonoscopy negative controls.

Phase I: Discovery of Differentially Expressed miRNAs Between Colorectal Adenomas and Carcinomas

Genome-wide NGS was carried out on 30 colorectal adenomas and 30 carcinoma frozen tissue specimens. The median age of the 30 patients with adenomas was 71.5 (range 48-82) and the median age of the colorectal carcinoma patients was 66.5 (range 47-88; supplementary table 1). NGS experiments were performed on 6 pools, each containing 500 ng of total RNA from 10 fresh frozen MSS colorectal adenomas or carcinomas (a total of 5 μg per pool).

Phase II: MiRNA Validation and miRNA Marker Selection in Tissue Samples

An independent collection of 150 frozen tissue samples including 55 patients with a colorectal carcinoma (median age 73, range 42-90), 73 colorectal adenoma patients (median age 65, range 40-90), and 22 normal colorectal tissue samples (median age 73, range 50-92; supplementary table 2) were used to verify the expression of the differentially expressed miRNAs, by RT-qPCR.

All frozen colorectal mucosae tissues (22 normal colorectal mucosa samples, 103 colorectal adenomas and 85 colorectal carcinomas) included in the study, were collected at the department of Pathology of the VU University medical center, Amsterdam (the Netherlands), between 1999 and 2011. All tumor samples were reviewed by an expert gastrointestinal pathologist (GAM). CRC samples were classified according to the TNM classification (fifth edition) and all adenoma samples were larger than 1 centimeter. All adenoma and carcinoma samples included a minimum of 70% tumor tissue and were stored at −80° C. until use. All included tissue samples were microsatellite stable (MSS; data were already available). Collection, storage and use of tissue and patient data were performed in compliance with the ‘Code for Proper Secondary Use of Human Tissue in the Netherlands’ (http://www.fmwv.nl) and approved in protocol 2012-71 of the department of Pathology, VU University medical center Amsterdam.

### Phase III: MiRNA Marker Validation in Stool Samples

The expression of the miRNAs differentially expressed in tissue samples was determined in 430 homogenized whole stool samples collected from 109 control individuals (median age 57, range 40-89), 55 non-advanced adenoma patients (median 64, range 42-89), 53 advanced adenoma patients (median 68, range 41-87) and 213 colorectal cancer patients (median age 70, range 34-89; table 1 and supplementary table 3). Stool samples from CRC patients diagnosed with all stages of CRC were collected at the VU University Medical Center in Amsterdam and from a multicenter prospective trial in Germany, in compliance with the institutional ethical regulations. Stool samples from control individuals and patients with colorectal adenomas were collected at the VU University Medical Center in Amsterdam, also in compliance with the institutional ethical regulations. Control stool samples were selected based on the absence of abnormalities as determined by colonoscopy, moderate to good bowel preparation, complete colonoscopy by means of reaching the cecum, no hereditary history of CRC or other cancers, and age 40. A total of 420 samples were collected befores colonoscopy (pre-colonoscopy samples) and 10 CRC stool samples were collected more than 2 weeks after colonoscopy (post-colonoscopy samples). Stool stabilization buffer (Exact Sciences, Madison, Wis., USA) was added to the stool samples immediately after defecation, processed in the laboratory with a final stool:buffer w/v ratio of 1:7 within 72 hours, and stored at −80° C. until use.

FIT (Fecal Immunochemical Test) data (OC Sensor, Eiken Chemical Co, Tokyo, Japan) were available for a subset of 179 stool samples 28, including 19 CRC patients, 37 advanced adenoma patients, 44 non-advanced adenoma patients and 79 control individuals.

### RNA Isolation

Total tissue RNA was isolated using TRIzol (Invitrogen, Carlsbad, Calif., USA) according to the manufacturer's guidelines with some modifications (Gastroenterology 2002; 123:1109-1119). Total tissue RNA was cleaned using RNeasy Mini kit (Qiagen, Hilden, Germany). Quantity was determined with a Nanodrop ND-1000 spectrophotometer (Isogen, Hackensack, N.J., USA) and quality was assessed on a 1% agarose gel, stained with ethidium bromide.

Total stool RNA was isolated using 6 ml of TRIzol (Invitrogen, Carlsbad, Calif., USA) on 2 ml of homogenized stool, following the manufacturer's protocol and subsequent cleanup was performed by ethanol precipitation. Briefly, the complete obtained volume of total stool RNA from the initial isolation was used, to which 0.1 times its volume of NaAc 3M (pH 5.2) and 2.5 times of the original volume of 100% EtOH were added. This mixture was spun down for 30 minutes at 13,000 rpm and 4° C. The supernatant was discarded and the air-dried pellet was resuspended in 50 μl of H2O. Concentrations were determined with a Nanodrop

Next Generation miRNA Sequencing

miRNA libraries were prepared by the Ambion SREK protocol, according to the manufacturer's guidelines with some modifications as previously described (Genome Res 2009; 19:2064-2074; BMC Genomics 2010; 11:249). An amount of 2.5 μg total RNA was used as starting material and half of the suggested amounts of reagents were used until PCR. Total RNA was size fractionated on a 15% PAA denaturing gel to collect small RNAs only. Directly after PCR, size selection for miRNAs from the amplified libraries was performed on a 6% denaturing PAA gel between 105 and 125 base pairs, comprising the miRNA with adaptor. DNA size and quantity were checked on a Bioanalyzer High Sensitivity chip. Library quality was checked by Sanger sequencing. Emulsion PCR and SOLiD sequencing (Applied Biosystems) were carried out as described before (Mol Cell 2007; 28:328-336): the read length was set to 30 nucleotides.

miRNA Expression by Real-Time RT-qPCR

A total of 22.5 ng total tissue RNA was reverse transcribed by use of the miRCURY LNATM Universal RT microRNA PCR system (Exiqon, Vedbaek, Denmark), according to the manufacturer's guidelines. Real-time qPCR amplification was performed using SYBR Green master mix with ROX passive reference dye and miRNA specific PCR primer assays (n=53, supplementary table 4), according to the manufacturer's protocol. All reactions were carried out in duplicate on a 7900 Fast Real-Time PCR system (Applied Biosystems, Foster City, Calif., USA). Hsa-miR-16 and hsa-miR-24 were used as endogenous references (BMC Cancer 2010; 10:173) and RNA from CRC cell line HT29 and a Universal Human Reference sample (Agilent Technologies, Santa Clara, Calif., USA), were used as positive controls. Briefly, the amplification protocol consisted of initial denaturation at 95° C. for 10 minutes, followed by 45 cycles for the tissue derived cDNA or 50 cycles for the stool derived cDNA, comprising a denaturation step at 95° C. for 10 seconds followed by annealing/elongation at 60° C. for 1 minute, completed by a melting curve analysis.

Potential housekeeping miRNAs were selected based on literature and our own NGS expression data. The selection criteria comprised; FDR=1, abundantly expressed in colon tissue, and measurable in stool. Housekeeping analysis was performed based on NormFinder (Cancer Res 2004; 64:5245-5250), which is an algorithm to identify the optimal normalization gene among a set of candidates. It ranks the candidate genes according to their expression stability, which is based on overall expression variation of the candidates and variation between sample subgroups of the sample set. Genes with the lowest stability value have the most stable expression.

### Statistical Analysis

The NGS data were first preprocessed by filtering out miRNAs with total count less than five and normalized by correcting for different library sizes. Then edgeR's exactTest (Bioinformatics 2007; 23:2881-2887) was used to determine discriminating miRNAs (Benjamini-Hochberg FDR<0.2). miRNA expression levels, determined by RT-qPCR, were calculated from the obtained C_{T} values using the 2^{-Δc}_{T} method (Methods 2001; 25:402-408). For tissue samples only measurements with ΔC_{T}<0.5 were considered proper measurements and miRNAs with less than half of the samples containing inadequate measurements (meaning either undetermined or ΔC_{T}>0.5) were taken along in the analyses. Stool samples showing a ΔC_{T}<2.55 and C_{T}<40 were considered adequate measurements. Samples with insufficient performance were repeatedly tested, with a maximum of two measurements and otherwise considered as poor quality and thus deleted from the analyses. Besides, samples lacking housekeeping miRNA expression (hsa-miR-24) were considered as poor RNA quality and were also discarded from the analyses.

Significance of differences in expression levels was computed by the Mann-Whitney U test for independent samples. All p-values were two-sided and p<0.05 was considered statistically significant. Performance of stool miRNAs in discriminating different sample groups was determined by development of a classification model, based on stepwise logistic regression (applying R functions glm and step). This renders a predicted probability to belong to one of the two classes, which is used as a variable threshold for the ROC curves. Statistical calculations were performed with R (versions 2.11.1 and 3.0.1) and SPSS software (SPSS 20.0 for Windows, SPSS Inc., Chicago, Ill., USA).

### Results

NGS Discovery Revealed 57 miRNAs to be Differentially Expressed Between Colorectal Adenomas and Carcinomas

Statistical analysis of the NGS genome-wide miRNA discovery study revealed 57 miRNAs to be differentially expressed between colorectal adenomas and carcinomas (FDR<0.2). Out of these, 23 known miRNAs were down-regulated in colorectal carcinomas compared to adenomas and 30 miRNAs were up-regulated in colorectal carcinomas compared to adenomas. In addition, three candidate small non-coding RNAs were down-regulated in colorectal carcinomas and one was up-regulated. Details of the differentially expressed miRNAs (miRNA name, chromosomal location, ratio, p-value and FDR) are provided in Table 5.

Twenty-One miRNAs were Independently Validated to be Differentially Expressed Between Colorectal Adenomas and Carcinomas

For independent validation, the expression of the 57 differentially expressed miRNAs in the NGS discovery study was further investigated by RT-qPCR in a set of 152 independent colorectal tissue specimens. Based on these NGS data and literature, two new housekeeping miRNAs were identified, which were specifically suitable for the validation and stool sample experiments. For the tissue samples hsa-miR-16 appeared to be the most stable expressed miRNA with a stability value of 0.016, compared to a stability value of 0.017 of RNU43, which is one of the established and well-accepted housekeeping genes for RT-qPCR. In stool samples hsa-miR-24 appeared to be the most stable miRNA with a stability value of 0.004, compared to a stability value of 0.015 of RNU43 in stool material.

By qRT-PCR two miRNAs showed significant differences in expression, but in opposite direction compared to the NGS data and were therefore not considered to validate the NGS results. For 21 miRNAs differential expression, normalized for hsa-miR-16 and p<0.05, was confirmed. Table 2 displays a complete list of the validated miRNAs: p-values from both NGS and RT-qPCR experiments, as well as the obtained ratios comparing expression in carcinomas with expression in adenomas also obtained by both NGS and RT-qPCR.

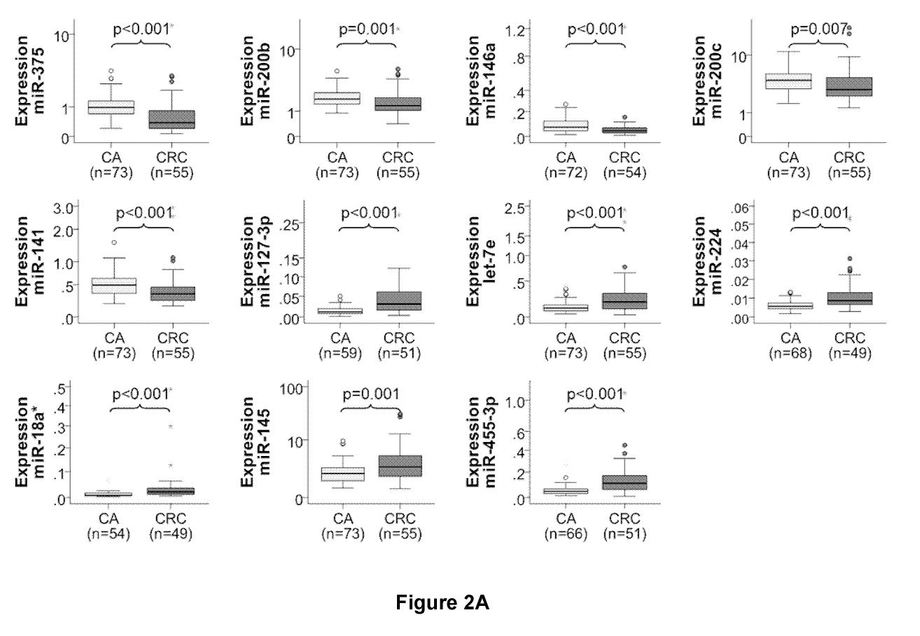

Of the 21 validated miRNAs, five were down-regulated in carcinomas compared to the adenomas, having fold changes ranging from 0.76 for hsa-miR-375 to 0.98 for hsa-miR-200b. A total of 16 miRNAs showed a higher expression in carcinomas compared to the adenomas, with fold changes ranging from 1.76 for hsa-miR-17 to 6.68 for hsa-miR-99a. Expression levels are visualized in the boxplots of FIG. 2.

Identification of miRNAs in Stool

For the 21 miRNAs differentially expressed between colorectal adenomas and carcinomas the technical performance of the assays was tested in a subset of 24 stool samples (eight control individuals, eight advanced adenoma patients and eight CRC patients). From the 21 selected miRNAs, a total of eight miRNAs were detected in stool; including hsa-miR-375, hsa-miR-200c, hsa-miR-200b, hsa-miR-141, hsa-miR-223, hsa-miR-455-3p, hsa-miR-214 and hsa-miR-146a.

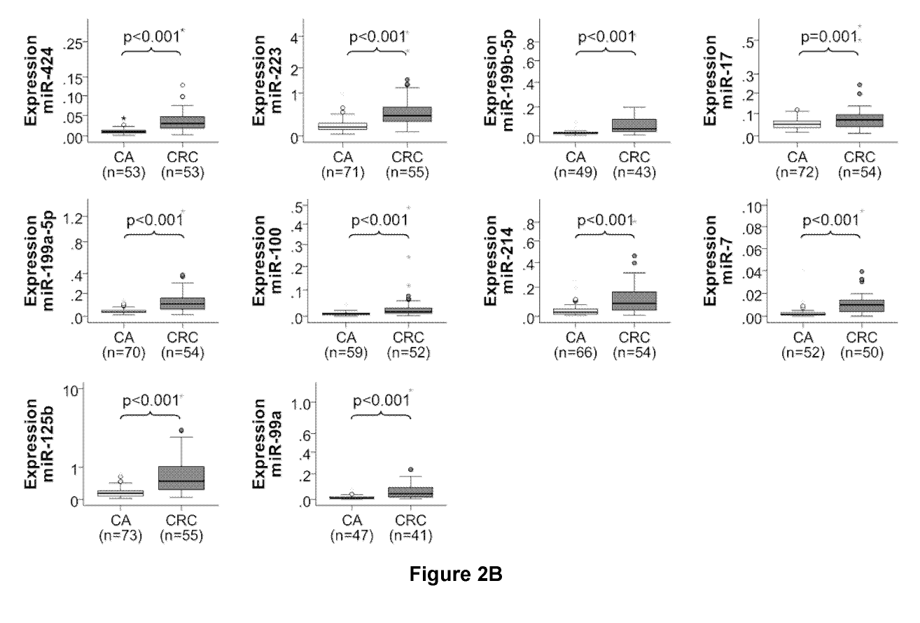

Levels of these eight selected miRNAs were determined in a large collection of stool samples from 213 colorectal cancer patients, 53 advanced adenoma patients, 55 non-advanced adenoma patients and 109 colonoscopy negative control individuals. Normalized expression levels (normalized for hsa-miR-24) are displayed in the boxplots of FIG. 3. After filtering for quality control criteria (housekeeping miRNA expression, ΔC_{T}<2.55 or C_{T}<40), a series of 110 CRC patients (51.6%), 39 advanced adenoma patients (73.6%), 42 non-advanced adenoma patients (76.4%) and 76 healthy control individuals (69.7%) remained.

Expression levels of hsa-miR-200b, hsa-miR-141, hsa-miR-223 and hsa-miR-214 were significantly different between stool from healthy individuals and colorectal cancer patients (p=0.01, p=0.005, p<0.001, p=0.03, respectively). Furthermore, for hsa-miR-223 significant differences between stage I CRC and all other CRC stages were found: p=0.03 for stage I versus stage II, p=0.02 for stage I versus stage III and p=0.03 for stage I versus stage IV. For hsa-miR-455-3p expression levels in stage I CRC were significantly different from those in stage III (p=0.02) and IV CRC (p=0.04).

Multivariate Analysis and Combination with FIT: miR-223 Accurately Discriminates Stool from CRC Patients and Healthy Individuals

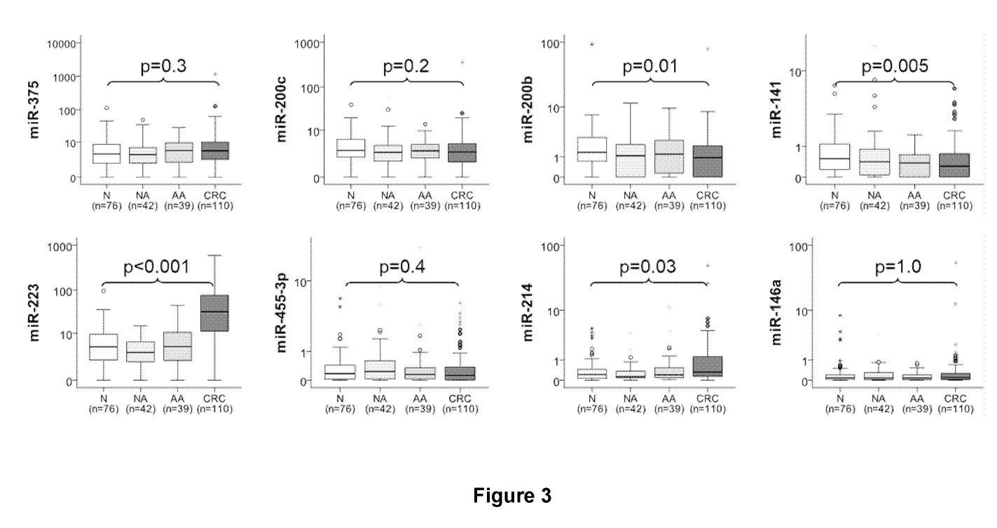

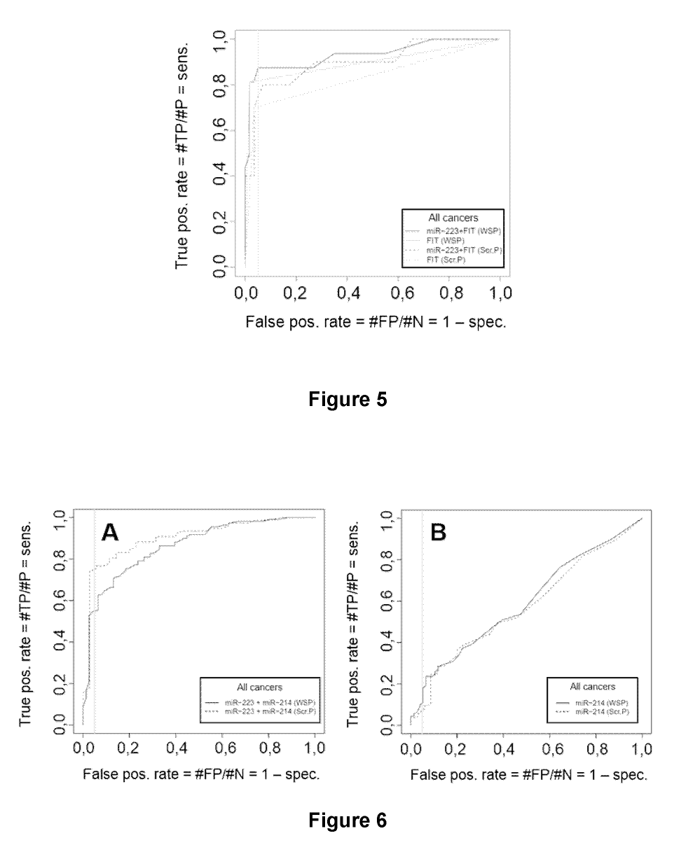

To determine the test accuracy of stool based miRNA expression, a step-wise regression model was applied. A combination of two up-regulated miRNAs in stool, i.e. hsa-miR-223 and hsa-miR-214, appeared to be the optimal combination to discriminate healthy controls from CRC patients. ROC analysis was performed for two defined populations: one includes the whole study population and the other ROC analysis includes the subset of individuals at screening age, i.e. 55 and 75 years (from now on referred to as screening age population). ROC analysis demonstrated for this particular model in the whole study population an AUC of 0.87, with 55% sensitivity at 95% specificity; in the screening age population an AUC of 0.90 was reached with 74% sensitivity at 95% specificity (FIG. 6). Performance of miR-223 alone also showed an excellent discrimination when comparing carcinoma patients with control individuals, in the whole study population with an AUC of 0.86, 51% sensitivity at 95% specificity, and in the screening age population an AUC of 0.90, 74% sensitivity at 95% specificity (FIG. 4A).

Since the combination of miR-223 and miR-214 expression showed limited added value to the performance of miR-223 alone, further analysis was focused on a model with miR-223 expression alone, looking at all cancers, stage I and II cancers, advanced adenomas and the combination of the latter two categories, also referred to as screening-relevant lesions. For discriminating CRC stage I and II from controls in the screening age population, miR-223 in stool reached at 95% specificity a 64% sensitivity (FIG. 4B). Adding advanced adenomas to this analysis resulted at 95% specificity at 43% sensitivity (FIG. 4D). By analyzing advanced adenomas alone, compared to controls, an AUC of 0.63 was reached, with 23% sensitivity at 95% specificity in the screening age population and 5% sensitivity at 95% specificity in the whole study population (FIG. 4C).

The currently most widely used stool test for bowel cancer screening is the hemoglobin based FIT. A subset of individuals from whom a whole stool sample was collected also performed a FIT, allowing to analyze the added value of miR-223 to FIT alone (n=179). At 95% specificity, FIT alone had a 82% sensitivity (AUC 0.90) in the whole study population, and 70% sensitivity (AUC 0.85) in the screening age population. Combining miR-223 with FIT showed at 95% specificity an increase to 88% sensitivity in the whole study population (AUC 0.94) and 73% sensitivity in the screening age population (AUC 0.91).

In general, total RNA isolation methods are applied in miRNA research, but more targeted isolation or detection methods may be necessary for future applications. On the other hand, large-scale testing is also essential, especially with respect to population screening tests, and that makes not every targeted method appropriate. A potential interesting technique may be QuARTS technology (Quantitative Allele-Specific Real-time Target and Signal amplification) (Clin Chem 2012; 58:375-383). This is a method similar to real-time PCR, with which it is possible to detect multiple (methylated) gene markers in a single assay. This technology reaches a high analytical sensitivity and therefore it may be very suitable for the detection of targets in stool, which come at lower detection rates.

In conclusion, based on a genome-wide approach, the current study demonstrated miR-223 to be differentially expressed between colorectal adenomas and cancer. In addition, it appeared to be detectable in stool, and as a biomarker it had additional value in discriminating colorectal cancer patients from healthy controls. As such, it holds promise as a marker for detecting colorectal cancer in a population-based screening program.

Any one or more features described for any aspect of the present invention or preferred embodiments or examples thereof, described herein, may be used in conjunction with any one or more other features described for any other aspect of the present invention or preferred embodiments or examples thereof described herein. The fact that a feature may only be described in relation to one aspect or embodiment or example does not limit its relevance to only that aspect or embodiment or example if it is technically relevant to one or more other aspect or embodiment or example.

While the methods and related aspects have been described with reference to certain examples, those skilled in the art will appreciate that various modifications, changes, omissions, and substitutions can be made without departing from the spirit of the disclosure. It is intended, therefore, that the invention be limited by the scope of the following claims. The features of any dependent claim may be combined with the features of any of the other dependent claims or any and/or any of the independent claims.

TABLE 1A
Patient characteristics from tissue samples used for NGS

| | Sample | | | | | Degree of | Degree of |
|---|---|---|---|---|---|---|---|
| | ID | Age | Gender | Lesion | Histology | dysplasia | differentiation |
| Pool A-1 | A1 | 82 | M | Adenoma | Tubular | Moderate | NA |
| | A2 | 73 | M | Adenoma | Tubular | Severe | NA |
| | A3 | 75 | M | Adenoma | Tubulovillous | Moderate | NA |
| | A4 | 70 | M | Adenoma | Tubulovillous | Severe | NA |
| | A5 | 82 | M | Adenoma | Tubulovillous | Mild | NA |
| | A6 | 77 | M | Adenoma | Tubulovillous | Severe | NA |
| | A7 | 61 | M | Adenoma | Tubular | Moderate | NA |
| | A8 | 79 | F | Adenoma | Villous | Moderate | NA |
| | A9 | 79 | F | Adenoma | Villous | Moderate | NA |
| | A10 | 79 | F | Adenoma | Tubulovillous | Severe | NA |
| Pool A-2 | A11 | 65 | F | Adenoma | Tubular | Mild | NA |
| | A12 | 62 | M | Adenoma | Tubulovillous | Moderate | NA |
| | A13 | 59 | M | Adenoma | Tubular | Moderate | NA |
| | A14 | 71 | F | Adenoma | Tubulovillous | Severe | NA |
| | A15 | 75 | F | Adenoma | Villous | Moderate | NA |
| | A16 | 49 | F | Adenoma | Tubular | Moderate | NA |
| | A17 | 65 | M | Adenoma | Tubular | Moderate | NA |
| | A18 | 65 | F | Adenoma | Tubulovillous | Moderate | NA |
| | A19 | 74 | F | Adenoma | Tubular | Moderate | NA |
| | A20 | 77 | M | Adenoma | Tubulovillous | Moderate | NA |
| Pool A-3 | A21 | 72 | M | Adenoma | Tubular | Moderate | NA |
| | A22 | 79 | F | Adenoma | Tubular | Mild | NA |
| | A23 | 60 | M | Adenoma | Tubulovillous | Moderate | NA |
| | A24 | 58 | M | Adenoma | Tubulovillous | Severe | NA |
| | A25 | 70 | M | Adenoma | ND | ND | NA |
| | A26 | 80 | M | Adenoma | Tubulovillous | Severe | NA |
| | A27 | 54 | M | Adenoma | Tubular | Moderate | NA |
| | A28 | 48 | M | Adenoma | Tubular | Moderate | NA |
| | A29 | 65 | M | Adenoma | tubulovillous | Severe | NA |
| | A30 | 75 | M | Adenoma | Tubulovillous | Moderate | NA |

F = female;
M = male;
NA = not applicable;
ND = no data available

TABLE 1B
Patient characteristics from tissue samples used for NGS

| | Sample | | | | | Degree of | Degree of |
|---|---|---|---|---|---|---|---|
| | ID | Age | Gender | Lesion | Histology | dysplasia | differentiation |
| Pool C-1 | C1 | 65 | F | Carcinoma | NA | NA | Moderate |
| | C2 | 88 | F | Carcinoma | NA | NA | Moderate |
| | C3 | 63 | M | Carcinoma | NA | NA | Moderate |
| | C4 | 84 | F | Carcinoma | NA | NA | Moderate |
| | C5 | 66 | F | Carcinoma | NA | NA | Moderate |
| | C6 | 69 | F | Carcinoma | NA | NA | Moderate |
| | C7 | 66 | M | Carcinoma | NA | NA | Moderate |
| | C8 | 80 | F | Carcinoma | NA | NA | Moderate |
| | C9 | 57 | F | Carcinoma | NA | NA | Well |
| | C10 | 80 | F | Carcinoma | NA | NA | Poor |
| Pool C-2 | C11 | 71 | F | Carcinoma | NA | NA | Well |
| | C12 | 66 | F | Carcinoma | NA | NA | Moderate |
| | C13 | 79 | F | Carcinoma | NA | NA | Moderate |
| | C14 | 63 | M | Carcinoma | NA | NA | Poor |
| | C15 | 57 | F | Carcinoma | NA | NA | Moderate |
| | C16 | 65 | F | Carcinoma | NA | NA | Moderate |
| | C17 | 65 | M | Carcinoma | NA | NA | Moderate |
| | C18 | 67 | M | Carcinoma | NA | NA | Moderate |
| | C19 | 53 | M | Carcinoma | NA | NA | Moderate |
| | C20 | 67 | F | Carcinoma | NA | NA | Moderate |
| Pool C-3 | C21 | 82 | F | Carcinoma | NA | NA | Moderate |
| | C22 | 61 | M | Carcinoma | NA | NA | Moderate |
| | C23 | 85 | F | Carcinoma | NA | NA | Moderate |
| | C24 | 47 | M | Carcinoma | NA | NA | Well |
| | C25 | 74 | F | Carcinoma | NA | NA | Poor |
| | C26 | 56 | F | Carcinoma | NA | NA | Moderate |
| | C27 | 70 | F | Carcinoma | NA | NA | Moderate |
| | C28 | 71 | F | Carcinoma | NA | NA | Well |
| | C29 | 75 | F | Carcinoma | NA | NA | Moderate |
| | C30 | 53 | F | Carcinoma | NA | NA | Moderate |

F = female;
M = male;
NA = not applicable;
ND = no data available

TABLE 2
Patient characteristics from tissue samples used for RT-qPCR

| | | | | | Degree | Degree of |
|---|---|---|---|---|---|---|
| Sample | | | | | of | differen- |
| ID | Age | Gender | Lesion | Histology | dysplasia | tiation |
| A1 | 55 | M | Adenoma | Tubular | Moderate | NA |
| A2 | 80 | M | Adenoma | Tubular | Moderate | NA |
| A3 | 75 | F | Adenoma | Tubulovillous | Moderate | NA |
| A4 | 59 | M | Adenoma | Tubular | Moderate | NA |
| A5 | 52 | M | Adenoma | Tubulovillous | Moderate | NA |
| A6 | 56 | F | Adenoma | Tubular | Moderate | NA |
| A7 | 45 | F | Adenoma | Tubular | Moderate | NA |
| A8 | 80 | F | Adenoma | Tubulovillous | Severe | NA |
| A9 | 67 | F | Adenoma | Tubular | Moderate | NA |
| A10 | 76 | M | Adenoma | Tubulovillous | Severe | NA |
| A11 | 70 | M | Adenoma | Tubular | Severe | NA |
| A12 | 70 | M | Adenoma | Tubulovillous | Severe | NA |
| A13 | 53 | M | Adenoma | Tubulovillous | Severe | NA |
| A14 | 48 | F | Adenoma | Tubulovillous | Moderate | NA |
| A15 | 59 | F | Adenoma | Tubulovillous | Severe | NA |
| A16 | 57 | M | Adenoma | Tubulovillous | Moderate | NA |
| A17 | 64 | F | Adenoma | Tubulovillous | Severe | NA |
| A18 | 78 | M | Adenoma | Tubulovillous | Severe | NA |
| A19 | 63 | M | Adenoma | Tubulovillous | Moderate | NA |
| A20 | 63 | M | Adenoma | Tubulovillous | Moderate | NA |
| A21 | 82 | F | Adenoma | Tubular | Moderate | NA |
| A22 | 58 | F | Adenoma | Tubulovillous | Moderate | NA |
| A23 | 41 | M | Adenoma | Tubulovillous | Moderate | NA |
| A24 | 65 | M | Adenoma | Villous | Severe | NA |
| A25 | 90 | F | Adenoma | Tubulovillous | Moderate | NA |
| A26 | 63 | M | Adenoma | Tubulovillous | Severe | NA |
| A27 | 84 | F | Adenoma | Tubulovillous | Severe | NA |
| A28 | 69 | M | Adenoma | Tubular | Moderate | NA |
| A29 | 66 | M | Adenoma | Tubulovillous | Severe | NA |
| A30 | 67 | M | Adenoma | Tubulovillous | Moderate | NA |
| A31 | 78 | F | Adenoma | Tubular | Severe | NA |
| A32 | 82 | M | Adenoma | Tubular | Severe | NA |
| A33 | 65 | F | Adenoma | Tubular | Moderate | NA |
| A34 | 55 | F | Adenoma | Tubular | Moderate | NA |
| A35 | 74 | M | Adenoma | Villous | Moderate | NA |
| A36 | 61 | F | Adenoma | Tubulovillous | Severe | NA |
| A37 | 74 | M | Adenoma | Tubular | Moderate | NA |
| A38 | 51 | M | Adenoma | Tubular | Moderate | NA |
| A39 | 57 | M | Adenoma | Tubulovillous | Severe | NA |
| A40 | 60 | M | Adenoma | Tubulovillous | Moderate | NA |
| A41 | 67 | M | Adenoma | Tubular | Moderate | NA |
| A42 | 56 | M | Adenoma | Tubular | Moderate | NA |
| A43 | 74 | M | Adenoma | Tubulovillous | Severe | NA |
| A44 | 86 | M | Adenoma | Tubulovillous | Moderate | NA |
| A45 | 87 | M | Adenoma | Tubular | Moderate | NA |
| A46 | 48 | M | Adenoma | Tubular | Moderate | NA |
| A47 | 59 | F | Adenoma | Tubular | Moderate | NA |
| A48 | 77 | F | Adenoma | Tubular | Moderate | NA |
| A49 | 77 | M | Adenoma | Tubular | Moderate | NA |
| A50 | 72 | F | Adenoma | Tubulovillous | Moderate | NA |
| A51 | 56 | M | Adenoma | Tubular | Mild | NA |
| A52 | 51 | M | Adenoma | Tubulovillous | Mild | NA |
| A53 | 58 | F | Adenoma | Tubulovillous | Severe | NA |
| A54 | 56 | M | Adenoma | Tubulovillous | Severe | NA |
| A55 | 40 | F | Adenoma | Tubular | Severe | NA |
| A56 | 52 | F | Adenoma | Tubulovillous | Moderate | NA |
| A57 | 65 | M | Adenoma | Tubular | Mild | NA |
| A58 | 56 | M | Adenoma | Tubulovillous | Severe | NA |
| A59 | 63 | F | Adenoma | Tubulovillous | Mild | NA |
| A60 | 70 | M | Adenoma | Tubulovillous | Moderate | NA |
| A61 | 78 | F | Adenoma | Tubulovillous | Mild | NA |
| A62 | 56 | M | Adenoma | Tubulovillous | Severe | NA |
| A63 | 83 | F | Adenoma | Tubular | Moderate | NA |
| A64 | 48 | M | Adenoma | Tubulovillous | Moderate | NA |
| A65 | 78 | M | Adenoma | Tubulovillous | Moderate | NA |
| A66 | 64 | M | Adenoma | Tubular | Moderate | NA |
| A67 | 74 | F | Adenoma | Tubulovillous | Severe | NA |
| A68 | 75 | M | Adenoma | Tubulovillous | Moderate | NA |
| A69 | 68 | M | Adenoma | Tubulovillous | Moderate | NA |
| A70 | 45 | F | Adenoma | Tubulovillous | ND | NA |
| A71 | 55 | M | Adenoma | Tubulovillous | Moderate | NA |
| A72 | 82 | F | Adenoma | Tubulovillous | Mild | NA |
| A73 | 78 | F | Adenoma | Tubulovillous | Moderate | NA |
| C1 | 82 | M | Carcinoma | NA | NA | Well |
| C2 | 59 | M | Carcinoma | NA | NA | Moderate |
| C3 | 60 | F | Carcinoma | NA | NA | Well |
| C4 | 67 | F | Carcinoma | NA | NA | Moderate |
| C5 | 62 | M | Carcinoma | NA | NA | Moderate |
| C6 | 78 | F | Carcinoma | NA | NA | Moderate |
| C7 | 65 | F | Carcinoma | NA | NA | Moderate |
| C8 | 42 | F | Carcinoma | NA | NA | Moderate |
| C9 | 58 | M | Carcinoma | NA | NA | Moderate |
| C10 | 56 | M | Carcinoma | NA | NA | Moderate |
| C11 | 60 | M | Carcinoma | NA | NA | Moderate |
| C12 | 62 | M | Carcinoma | NA | NA | Moderate |
| C13 | 60 | M | Carcinoma | NA | NA | Moderate |
| C14 | 79 | M | Carcinoma | NA | NA | ND |
| C15 | 48 | M | Carcinoma | NA | NA | Moderate |
| C16 | 74 | F | Carcinoma | NA | NA | Moderate |
| C17 | 71 | F | Carcinoma | NA | NA | Poor |
| C18 | 76 | M | Carcinoma | NA | NA | Moderate |
| C19 | 75 | F | Carcinoma | NA | NA | Moderate |
| C20 | 82 | M | Carcinoma | NA | NA | Moderate |
| C21 | 76 | M | Carcinoma | NA | NA | Poor |
| C22 | 72 | F | Carcinoma | NA | NA | Moderate |
| C23 | 79 | F | Carcinoma | NA | NA | ND |
| C24 | 62 | F | Carcinoma | NA | NA | Moderate |
| C25 | 79 | M | Carcinoma | NA | NA | Moderate |
| C26 | 81 | M | Carcinoma | NA | NA | Moderate |
| C27 | 86 | F | Carcinoma | NA | NA | Moderate |
| C28 | 62 | M | Carcinoma | NA | NA | Moderate |
| C29 | 50 | F | Carcinoma | NA | NA | Moderate |
| C30 | 72 | M | Carcinoma | NA | NA | Moderate |
| C31 | 63 | M | Carcinoma | NA | NA | Poor |
| C32 | 88 | M | Carcinoma | NA | NA | Moderate |
| C33 | 83 | M | Carcinoma | NA | NA | Well |
| C34 | 58 | M | Carcinoma | NA | NA | Well |
| C35 | 86 | M | Carcinoma | NA | NA | Moderate |
| C36 | 74 | M | Carcinoma | NA | NA | Moderate |
| C37 | 90 | F | Carcinoma | NA | NA | ND |
| C38 | 58 | M | Carcinoma | NA | NA | Moderate |
| C39 | 81 | M | Carcinoma | NA | NA | Moderate |
| C40 | 78 | F | Carcinoma | NA | NA | Moderate |
| C41 | 73 | M | Carcinoma | NA | NA | Moderate |
| C42 | 67 | M | Carcinoma | NA | NA | Moderate |
| C43 | 71 | M | Carcinoma | NA | NA | Poor |
| C44 | 56 | M | Carcinoma | NA | NA | Poor |
| C45 | 67 | F | Carcinoma | NA | NA | Moderate |
| C46 | 73 | M | Carcinoma | NA | NA | Moderate |
| C47 | 85 | M | Carcinoma | NA | NA | Moderate |
| C48 | 74 | M | Carcinoma | NA | NA | Moderate |
| C49 | 72 | M | Carcinoma | NA | NA | Moderate |
| C50 | 84 | F | Carcinoma | NA | NA | Moderate |
| C51 | 88 | M | Carcinoma | NA | NA | Moderate |
| C52 | 74 | M | Carcinoma | NA | NA | Moderate |
| C53 | 75 | F | Carcinoma | NA | NA | Moderate |
| C54 | 80 | M | Carcinoma | NA | NA | Moderate |
| C55 | 61 | F | Carcinoma | NA | NA | Moderate |
| N1 | 91 | F | Normal | NA | NA | NA |
| N2 | 62 | M | Normal | NA | NA | NA |
| N3 | 77 | M | Normal | NA | NA | NA |
| N4 | 60 | M | Normal | NA | NA | NA |
| N5 | 73 | M | Normal | NA | NA | NA |
| N6 | 74 | F | Normal | NA | NA | NA |
| N7 | 78 | M | Normal | NA | NA | NA |
| N8 | 50 | F | Normal | NA | NA | NA |
| N9 | 71 | M | Normal | NA | NA | NA |
| N10 | 87 | F | Normal | NA | NA | NA |
| N11 | 74 | M | Normal | NA | NA | NA |
| N12 | 92 | F | Normal | NA | NA | NA |
| N13 | 79 | F | Normal | NA | NA | NA |
| N14 | 56 | M | Normal | NA | NA | NA |
| N15 | 72 | M | Normal | NA | NA | NA |
| N16 | 68 | M | Normal | NA | NA | NA |
| N17 | 89 | M | Normal | NA | NA | NA |
| N18 | 73 | M | Normal | NA | NA | NA |
| N19 | 72 | F | Normal | NA | NA | NA |
| N20 | 56 | M | Normal | NA | NA | NA |
| N21 | 87 | F | Normal | NA | NA | NA |
| N22 | 51 | F | Normal | NA | NA | NA |

F = female;
M = male;
NA = not applicable;
ND = no data available

TABLE 3
Overview of the clinicopathology of whole stool study population

| | | | Non- | | | | | |
|---|---|---|---|---|---|---|---|---|
| | | | advanced | | Advanced | | Colorectal | |
| Category | Normal | | adenoma | | adenoma | | cancer | |
| Number | 109 | | 55 | | 53 | | 213 | |
| of cases | | | | | | | | |
| Median age | 57 | (±12.4) | 64 | (±10.7) | 68 | (±10.5) | 70 | (±10.5) |
| (±SD) | | | | | | | | |
| Gender | | | | | | | | |
| Male | 42 | (38.5) | 35 | (63.6) | 25 | (47.2) | 129 | (60.6) |
| Female | 67 | (61.5) | 20 | (36.4) | 28 | (52.8) | 84 | (39.4) |
| Localisation | | | | | | | | |
| Left-sided | NA | | 33 | (60.6) | 34 | (64.2) | 19 | (8.9) |
| Right-sided | NA | | 15 | (27.3) | 13 | (24.5) | 10 | (4.7) |
| Complete | NA | | 6 | (10.9) | 6 | (11.3) | 0 | (0.0) |
| colon | | | | | | | | |
| ND | | | 1 | (1.8) | 0 | (0.0) | 184 | (86.4) |
| Histology | | | | | | | | |
| Tubular | NA | | 26 | (47.3) | 10 | (18.9) | NA | |
| Tubulovil- | NA | | 0 | (0.0) | 24 | (45.3) | NA | |
| lous | | | | | | | | |
| Villous | NA | | 0 | (0.0) | 2 | (3.8) | NA | |
| Serrated | NA | | 1 | (1.8) | 0 | (0.0) | NA | |
| Hyperplastic | NA | | 15 | (27.3) | 3 | (5.7) | NA | |
| ND | | | 13 | (23.6) | 13 | (24.5) | | |
| TNM stage | | | | | | | | |
| I | NA | | NA | | NA | | 21 | (9.9) |
| II | NA | | NA | | NA | | 41 | (19.2) |
| III | NA | | NA | | NA | | 103 | (48.4) |
| IV | NA | | NA | | NA | | 40 | (18.8) |
| ND | | | | | | | 8 | (3.8) |
| FIT score | | | | | | | | |
| FIT− | 77 | (70.6) | 37 | (67.3) | 21 | (39.6) | 4 | (1.9) |
| FIT+ | 2 | (1.8) | 7 | (12.7) | 16 | (30.2) | 15 | (7.0) |
| ND | 30 | (27.5) | 11 | (20.0) | 16 | (30.2) | 194 | (91.1) |

All numbers represent absolute numbers, between brackets percentages;
NA = not applicable;
ND = no data available

TABLE 4
miRNA assay details used in RT-qPCR validation

| | | Chromosomal | Assay | |
|---|---|---|---|---|
| | MiRNA assays | location | ID | Category |
| 1 | hsa-miR-320d 3 arm | 13q, Xq | custom | NGS hit |
| 2 | hsa-miR-345 5 arm | 14q | 204589 | NGS hit |
| 3 | hsa-miR-214 3 arm | 1q | 204510 | NGS hit |
| 4 | hsa-miR-503 5 arm | Xq | 204334 | NGS hit |
| 5 | hsa-miR-23b 5 arm | 9q | 204790 | NGS hit |
| 6 | hsa-miR-1274b 5 arm | 19q | custom | NGS hit |
| 7 | hsa-miR-18a 3 arm | 13q | 204523 | NGS hit |
| 8 | hsa-miR-874 3 arm | 5q | 204761 | NGS hit |
| 9 | hsa-miR-424 5 arm | Xq | 204736 | NGS hit |
| 10 | hsa-miR-767 5 arm | Xq | 204238 | NGS hit |
| 11 | hsa-miR-675 3 arm | 11p | 204732 | NGS hit |
| 12 | hsa-miR-199b 5 arm | 9q | 204152 | NGS hit |
| 13 | hsa-miR-494 3 arm | 14q | 204579 | NGS hit |
| 14 | hsa-miR-382 5 arm | 14q | 204169 | NGS hit |
| 15 | hsa-miR-105 5 arm | Xq | 204389 | NGS hit |
| 16 | hsa-miR-453 3 arm | Merged with miR-323b | | NGS hit |
| 17 | hsa-miR-199a 5 arm | 19p, 1q | 204494 | NGS hit |
| 18 | hsa-miR-125b 5 arm | 11q, 21q | 204465 | NGS hit |
| 19 | hsa-miR-542 3 arm | Xq | custom | NGS hit |
| 20 | block5519709_cand 5 | 6p | custom | NGS hit |
| | arm | | | |
| 21 | hsa-miR-155 3 arm | 21q | 204000 | NGS hit |
| 22 | hsa-miR-331 3 arm | 12q | 204156 | NGS hit |
| 23 | hsa-miR-146a 5 arm | 5q | 204688 | NGS hit |
| 24 | hsa-miR-99a 5 arm | 21q | 204521 | NGS hit |
| 25 | hsa-miR-455 3 arm | 9q | 204035 | NGS hit |
| 26 | hsa-miR-1280 5 arm | 3q | custom | NGS hit |
| 27 | hsa-miR-320b 3 arm | 1p, 1q | 204524 | NGS hit |
| 28 | hsa-miR-17 5 arm | 13q | 204771 | NGS hit |
| 29 | hsa-miR-320c 3 arm | 18q | custom | NGS hit |
| 30 | hsa-let-7e 5 arm | 19q | 204428 | NGS hit |
| 31 | hsa-miR-93 5 arm | 7q | 204715 | NGS hit |
| 32 | hsa-miR-660 3 arm | Xp | custom | NGS hit |
| 33 | hsa-miR-223 3 arm | Xq | 204256 | NGS hit |
| 34 | hsa-miR-204 5 arm | 9q | 204507 | NGS hit |
| 35 | hsa-miR-129 3 arm | 7q, 11p | 204058 | NGS hit |
| 36 | block5907700_cand 5 | 7q | custom | NGS hit |
| | arm | | | |
| 37 | hsa-miR-720 5 arm | 3q | 204088 | NGS hit |
| 38 | hsa-miR-127 3 arm | 14q | 204048 | NGS hit |
| 39 | hsa-miR-141 3 arm | 12p | 204504 | NGS hit |
| 40 | hsa-miR-1826 3 arm | Fragment of 5.8S rRNA | | NGS hit |
| 41 | hsa-miR-1 3 arm | 20q | 204344 | NGS hit |
| 42 | hsa-miR-375 5 arm | 2q | 204362 | NGS hit |
| 43 | hsa-miR-1978 3arm | Overlaps Mt tRNA | | NGS hit |
| 44 | hsa-miR-451 5 arm | 17q | 204734 | NGS hit |
| 45 | hsa-miR-200c 3 arm | 12p | 204482 | NGS hit |
| 46 | hsa-miR-7 5 arm | 9q, 15q, 19p | 204592 | NGS hit |
| 47 | blo ck2071264_cand 5 | 17q | custom | NGS hit |
| | arm | | | |
| 48 | hsa-miR-100 5 arm | 11q | 204133 | NGS hit |
| 49 | hsa-miR-200b 3 arm | 1p | 205111 | NGS hit |
| 50 | hsa-miR-224 5 arm | Xq | 204641 | NGS hit |
| 51 | hsa-miR-145 5 arm | 5q | 204483 | NGS hit |
| 52 | block5648011_cand 5 | 7p | custom | NGS hit |
| | arm | | | |
| 53 | hsa-miR-105 3 arm | Xq | 204193 | NGS hit |
| 54 | hsa-miR-1257 5 arm | 20q | custom | NGS hit |
| 55 | hsa-miR-23a 5 arm | 19p | 204559 | NGS hit |
| 56 | hsa-miR-493 5 arm | 14q | 204166 | NGS hit |
| 57 | hsa-miR-1268 5 arm | 15q | custom | NGS hit |
| 58 | hsa-miR-16 5 arm | 3q, 13q | 204409 | Housekeeping |
| | | | | miRNA |
| 59 | hsa-miR-24 3 arm | 19p, 19q | 204260 | Housekeeping |
| | | | | miRNA |

TABLE 5
Top 57 differentially expressed miRNAs between colorectal
adenomas and carcinomas by NGS (FDR < 0.2)

| | | Average | Average | | | |
|---|---|---|---|---|---|---|
| | | number | number | | | |
| | | of reads | of reads | Ratio | | |
| MiRNA | Location | adenomas | carcinomas | Ca/Ad | P-value | FDR |
| hsa-miR-320d 3 arm | 13q, Xq | 473.40 | 233.70 | 0.49 | 0.0000 | 0.0000 |
| hsa-miR-345 5 arm | 14q | 104.04 | 58.16 | 0.56 | 0.0000 | 0.0000 |
| hsa-miR-214 3 arm | 1q | 346.16 | 1006.46 | 2.91 | 0.0000 | 0.0000 |
| hsa-miR-503 5 arm | Xq | 12.09 | 34.21 | 2.83 | 0.0000 | 0.0000 |
| hsa-miR-23b 5 arm | 9q | 25.99 | 6.95 | 0.27 | 0.0000 | 0.0001 |
| hsa-miR-1274b 5 arm | 19q | 32.67 | 1.96 | 0.06 | 0.0000 | 0.0001 |
| hsa-miR-18a 3 arm | 13q | 26.65 | 52.18 | 1.96 | 0.0000 | 0.0009 |
| hsa-miR-874 3 arm | 5q | 23.64 | 7.88 | 0.33 | 0.0000 | 0.0022 |
| hsa-miR-424 5 arm | Xq | 15.05 | 34.12 | 2.27 | 0.0000 | 0.0032 |
| hsa-miR-767 5 arm | Xq | 0.17 | 12.85 | 74.69 | 0.0001 | 0.0035 |
| hsa-miR-675 3 arm | 11p | 0.17 | 7.71 | 44.80 | 0.0003 | 0.0162 |
| hsa-miR-199b 5 arm | 9q | 144.18 | 368.51 | 2.56 | 0.0004 | 0.0187 |
| hsa-miR-494 3 arm | 14q | 16.87 | 43.64 | 2.59 | 0.0004 | 0.0197 |
| hsa-miR-382 5 arm | 14q | 18.30 | 44.52 | 2.43 | 0.0005 | 0.0231 |
| hsa-miR-105 5 arm | Xq | 0.00 | 6.92 | | 0.0006 | 0.0238 |
| hsa-miR-453 3 arm | Merged | 0.00 | 4.54 | | 0.0007 | 0.0266 |
| | with | | | | | |
| | miR-323b | | | | | |
| hsa-miR-199a 5 arm | 19p, 1q | 390.81 | 1071.05 | 2.74 | 0.0007 | 0.0269 |
| hsa-miR-125b 5 arm | 11q, 21q | 486.36 | 1468.82 | 3.02 | 0.0010 | 0.0342 |
| hsa-miR-542 3 arm | Xq | 0.77 | 6.96 | 9.06 | 0.0010 | 0.0342 |
| Block5519709_cand | 6p | 6.34 | 0.00 | 0.00 | 0.0011 | 0.0345 |
| 5 arm | | | | | | |
| hsa-miR-155 3 arm | 21q | 8.22 | 1.42 | 0.17 | 0.0012 | 0.0345 |
| hsa-miR-331 3 arm | 12q | 747.76 | 411.50 | 0.55 | 0.0012 | 0.0345 |
| hsa-miR-146a 5 arm | 5q | 1611.15 | 650.40 | 0.40 | 0.0013 | 0.0345 |
| hsa-miR-99a 5 arm | 21q | 110.50 | 385.20 | 3.49 | 0.0014 | 0.0376 |
| hsa-miR-455 3 arm | 9q | 449.73 | 897.33 | 2.00 | 0.0019 | 0.0480 |
| hsa-miR-1280 5 arm | 3q | 16.72 | 2.38 | 0.14 | 0.0020 | 0.0480 |
| hsa-miR-320b 3 arm | 1p, 1q | 837.04 | 406.57 | 0.49 | 0.0027 | 0.0628 |
| hsa-miR-17 5 arm | 13q | 1430.32 | 3847.69 | 2.69 | 0.0030 | 0.0632 |
| hsa-miR-320c 3 arm | 18q | 362.14 | 185.03 | 0.51 | 0.0030 | 0.0632 |
| hsa-let-7e 5 arm | 19q | 304.78 | 495.78 | 1.63 | 0.0031 | 0.0632 |
| hsa-miR-93 5 arm | 7q | 90.69 | 189.02 | 2.08 | 0.0032 | 0.0632 |
| hsa-miR-660 3 arm | Xp | 39.08 | 24.20 | 0.62 | 0.0032 | 0.0632 |
| hsa-miR-223 3 arm | Xq | 1237.03 | 2810.35 | 2.27 | 0.0034 | 0.0644 |
| hsa-miR-204 5 arm | 9q | 2.65 | 29.22 | 11.02 | 0.0035 | 0.0647 |
| hsa-miR-129 3 arm | 7q, 11p | 0.40 | 8.09 | 20.01 | 0.0037 | 0.0675 |
| Block5907700_cand | 7q | 7.74 | 0.00 | 0.00 | 0.0039 | 0.0685 |
| 5 arm | | | | | | |
| hsa-miR-720 5 arm | 3q | 11.98 | 1.65 | 0.14 | 0.0041 | 0.0697 |
| hsa-miR-127 3 arm | 14q | 42.46 | 68.49 | 1.61 | 0.0042 | 0.0697 |
| hsa-miR-141 3 arm | 12p | 254.12 | 136.56 | 0.54 | 0.0046 | 0.0738 |
| hsa-miR-1826 3 arm | Fragment | 22.92 | 7.01 | 0.31 | 0.0059 | 0.0923 |
| | of 5.8 S | | | | | |
| | rRNA | | | | | |
| hsa-miR-1 3 arm | 20q | 3.36 | 38.14 | 11.37 | 0.0060 | 0.0923 |
| hsa-miR-375 5 arm | 2q | 7.57 | 1.51 | 0.20 | 0.0061 | 0.0923 |
| hsa-miR-1978 3 arm | Overlaps | 99.94 | 49.51 | 0.50 | 0.0064 | 0.0941 |
| | Mt tRNA | | | | | |
| hsa-miR-451 5 arm | 17q | 2452.83 | 808.00 | 0.33 | 0.0080 | 0.1146 |
| hsa-miR-200c 3 arm | 12p | 4493.93 | 2181.56 | 0.49 | 0.0095 | 0.1337 |
| hsa-miR-7 5 arm | 9q, 15q, | 82.40 | 245.04 | 2.97 | 0.0101 | 0.1382 |
| | 19p | | | | | |
| Block2071264_cand | 17q | 2.89 | 0.00 | 0.00 | 0.0103 | 0.1382 |
| 5 arm | | | | | | |
| hsa-miR-100 5 arm | 11q | 39.72 | 112.35 | 2.83 | 0.0112 | 0.1478 |
| hsa-miR-200b 3 arm | 1p | 7414.47 | 2951.40 | 0.40 | 0.0122 | 0.1572 |
| hsa-miR-224 5 arm | Xq | 884.29 | 1497.23 | 1.69 | 0.0129 | 0.1624 |
| hsa-miR-145 5 arm | 5q | 16342.97 | 32581.24 | 1.99 | 0.0131 | 0.1624 |
| Block5648011_cand | 7p | 0.36 | 4.07 | 11.23 | 0.0137 | 0.1666 |
| 5 arm | | | | | | |
| hsa-miR-105 3 arm | Xq | 0.00 | 3.88 | | 0.0140 | 0.1666 |
| hsa-miR-1257 5 arm | 20q | 17.94 | 6.67 | 0.37 | 0.0147 | 0.1720 |
| hsa-miR-23a 5 arm | 19p | 38.77 | 15.40 | 0.40 | 0.0153 | 0.1745 |
| hsa-miR-493 5 arm | 14q | 10.83 | 19.21 | 1.77 | 0.0155 | 0.1745 |
| hsa-miR-1268 5 arm | 15q | 12.56 | 5.83 | 0.46 | 0.0162 | 0.1793 |

1. A method of detecting colorectal cancer or a precursor of colorectal cancer in a subject, the method comprising:
obtaining a stool sample from the subject;
analyzing the stool sample for the increased expression of hsa-miR-223 relative to a reference sample, wherein the increased expression of hsa-miR-223 relative to the reference sample is indicative that the individual is suffering from colorectal cancer or a precursor of colorectal cancer.

2. The method of claim 1, wherein the subject has or is suspected of having adenoma.

3. The method of claim 1, wherein the subject is suspected of having colorectal cancer.

4. The method of claim 1, wherein the reference sample is from an individual not having colorectal cancer, or from an individual with non-advanced adenoma, or from an individual with advanced adenoma.

5. The method of claim 1, wherein the increased expression of hsa-miR-223 is determined using a binding agent that selectively binds hsa-miR-223.

6. The method of claim 5, wherein the binding agent is an antibody, an aptamer or at least one complementary nucleic acid sequence.

7. The method of claim 6, wherein the at least one complementary nucleic acid sequence comprises a pair of PCR primers specific to hsa-miR-223.

8. The method of claim 1, wherein the expression level of hsa-miR-223 is determined using mass spectrometry.

9. The method of claim 1, wherein the increased expression of hsa-miR-223 is determined using RT-qPCR.

10. The method of claim 1, wherein the increased expression of hsa-miR-223 is determined by sequencing the total RNA of the sample and determining the expression level of hsa-miR-223.

11. The method of claim 10, wherein the total RNA is subjected to size selection of small RNAs before sequencing.

12. The method of claim 1, wherein the method further comprises analyzing the stool sample using the fecal immunochemical test.

13. The method of claim 1, wherein the stool sample is prepared for analysis by extracting the total RNA from the sample.

14. The method of claim 1, further comprising analyzing the stool sample for the expression levels of one or more of hsa-miR-375, hsa-miR-200c, hsa-miR-200b, hsa-miR-141, hsa-miR-455-3p, hsa-miR-214 and hsa-miR-146a.

15. (canceled)

16. (canceled)

17. A kit for measuring the level of hsa-miR-223 in a stool sample obtained from a subject, the kit comprising: (a) an hsa-miR-223 binding agent that selectively binds hsa-miR-223, (b) packaging materials and instructions for measuring hsa-miR-223 in a sample, the kit being present in association with instructions for use according to the method of claim 1.

18. The kit of claim 17, further comprising binding agents that selectively bind one or more of hsa-miR-375, hsa-miR-200c, hsa-miR-200b, hsa-miR-141, hsa-miR-455-3p, hsa-miR-214 and hsa-miR-146a.

19. An array for detecting colorectal cancer, the array comprising a binding agent specific to hsa-miR-223.

20. The array of claim 19, further comprising binding agents specific to one or more of hsa-miR-375, hsa-miR-200c, hsa-miR-200b, hsa-miR-141, hsa-miR-455-3p, hsa-miR-214 and hsa-miR-146a.

21. (canceled)

22. (canceled)


SEQUENCE LISTING (9 sequences)

SEQ ID NO: 1 - RNA, 109 nt, Homo sapiens
ccuggccuccugcagugccacgcuccguguauuugacaagcugaguuggacacuccauggguagagugucaguuugucaaauaccccaagugcggcacaugcuuaccag

SEQ ID NO: 2 - DNA, 22 bp, Artificial Sequence
  PCR Primer
tggggtatttgacaaactgaca

SEQ ID NO: 3 - DNA, 22 bp, Artificial Sequence
  PCR Primer
aactcagcttgtcaaatacacg

SEQ ID NO: 4 - DNA, 22 bp, Artificial Sequence
  PCR Primer
tcatcattaccaggcagtatta

SEQ ID NO: 5 - DNA, 22 bp, Artificial Sequence
  PCR Primer
tccaatgctgcccagtaagatg

SEQ ID NO: 6 - DNA, 22 bp, Artificial Sequence
  PCR Primer
ccatctttaccagacagtgtta

SEQ ID NO: 7 - DNA, 22 bp, Artificial Sequence
  PCR Primer
tccaacactgtactggaagatg

SEQ ID NO: 8 - DNA, 22 bp, Artificial Sequence
  PCR Primer
actgcctgtctgtgcctgctgt

SEQ ID NO: 9 - DNA, 22 bp, Artificial Sequence
  PCR Primer
gcacagcaagtgtagacaggca